(19) United States
(12) Patent Application Publication
(10) Pub. No.: US 2012/0115812 A1
(43) Pub. Date: May 10, 2012

(54) SURFACE COATINGS FOR SKIN

(75) Inventor: James Donald Hammer, Uxbridge, MA (US)

(73) Assignee: Pharmasol Corporation, South Easton, MA (US)

(21) Appl. No.: 13/351,878

(22) Filed: Jan. 17, 2012

Related U.S. Application Data
(63) Continuation-in-part of application No. 12/890,187, filed on Sep. 24, 2010.
(60) Provisional application No. 61/245,888, filed on Sep. 25, 2009.

(51) Int. Cl. A61K 31/695; A61P 17/00; A61K 31/075; A61K 47/30; A61K 31/14

(52) U.S. Cl. 514/63; 514/772.3; 514/643; 514/721

(57) ABSTRACT

The present invention relates to skin coating compositions and methods and, in particular, to coatings containing an active agent. In some embodiments, the coating may be applied to a skin surface and may be essentially colorless and transparent. In some aspects, the coating comprises a polymer and an active agent, and the polymer may be configured for sustained release of the active agent. In some aspects, the coating forms a barrier essentially impermeable to microorganisms yet permeable to water vapor. The coating may also be flexible and conformable to the surface. The coating may be applied to the skin using any suitable method. In some embodiments, the polymer and/or active agent are dissolved and/or suspended in a volatile solvent. In some embodiments, the volatile solvent may function as an antiseptic on skin.

### FIELD OF INVENTION

The present invention relates to skin coating compositions and methods of application thereof and, in particular, to coatings containing an active agent.

### BACKGROUND

Coatings applied to a skin surface may perform a variety of short term and/or long term functions (i.e., prophylactic and/or drug delivery) and can provide a convenient medium from which to achieve such functionality rapidly. Compositions and methods known in the art include those described in U.S. Pat. Nos. 2,804,073 (Gallienne et al.); 4,379,863 (Bard); 4,542,012 (Dell); 6,228,354 (Jeng); and 6,613,755 (Peterson et al.).

### SUMMARY OF THE INVENTION

The present invention relates to skin coating compositions and methods of application thereof and, in particular, to coatings containing an active agent. The subject matter of the present invention involves, in some embodiments, interrelated products, alternative solutions to a particular problem, and/or a plurality of different uses of one or more compositions and/or methods.

In one aspect, a composition is provided. The composition comprises an essentially water-insoluble polymer, an agent, and a volatile solvent, wherein the polymer and agent form an essentially colorless and transparent coating on a surface upon evaporation of the volatile solvent, wherein the coating is essentially impermeable to microorganisms, permeable to moisture vapor, and configured for controlled release of the agent, wherein the volatile solvent is initially present at a concentration between 60% and 95% by volume.

In another aspect, a composition is provided. The composition comprises an essentially water-insoluble polymer, an agent, a quaternary amine antiseptic, and a volatile solvent, wherein the polymer and agent form an essentially colorless and transparent coating on a surface upon evaporation of the volatile solvent, wherein the coating is essentially impermeable to microorganisms, permeable to moisture vapor, and configured for controlled release of the agent, wherein the volatile solvent is initially present at a concentration less than 60% by volume.

In yet another aspect, a method is provided. The method comprises contacting a surface with a composition comprising a polymer, an active agent, and a volatile solvent, wherein the polymer and active agent form an essentially colorless and transparent coating on the surface upon evaporation of the volatile solvent, the composition sanitizes the surface essentially immediately, and the coating is capable of sustained antisepsis.

The present invention also relates to pharmaceutical compositions comprising any of the compositions described above and herein, as well as one or more pharmaceutically acceptable carriers, additives, and/or diluents.

### DETAILED DESCRIPTION OF THE INVENTION

The present invention relates to skin coating compositions and methods of application thereof and, in particular, to coatings containing an active agent. In some embodiments, the coating may be applied to a skin surface and may be essentially colorless and transparent. In some aspects, the coating comprises a polymer and an active agent, and the polymer may be configured for sustained release of the active agent. In some aspects, the coating forms a barrier essentially impermeable to microorganisms yet permeable to water vapor. The coating may also be flexible and conformable to the surface. The coating may be applied to the skin using any suitable method. In some embodiments, the polymer and/or active agent are dissolved and/or suspended in a volatile solvent. In some embodiments, the volatile solvent may function as an antiseptic on skin.

Skin may be subject to deleterious events such as microbial and/or viral contamination, fouling, soiling, damage, injury, etc. and/or may have inferior properties such as slipperiness, fragility, sensitivity (i.e., to light damage, oxidation, aging, etc.), and the like. One way to modify skin to mitigate undesirable properties, protect the skin, and/or add new properties is to apply a coating on the skin surface. The coating may serve as a barrier that can remain on the skin surface for an extended period of time (i.e., 24 hours or more) or until deliberately removed, which, in some embodiments and as described in more detail below, may protect the surface. In embodiments where the coating contains an agent, the coating may allow an agent to remain in contact with the skin. As discussed in more detail below, the coating may serve as a barrier to protect the skin, and may be used to deliver an agent to the skin surface beneath the coating and/or to the coating surface.

Certain existing coatings are deficient in that they discolor the surface on which they are applied, do not possess essentially immediate antiseptic properties, do not contain an agent that is capable of a sustained effect, are not permeable to water vapor, and/or are not resilient. Thus, a non-limiting aim of certain embodiments described herein is to provide a coating composition that forms a protective barrier on a surface, possesses essentially immediate antiseptic properties, and possesses sustained antiseptic properties.

Advantageously, compositions and methods of the invention can be used in a variety of applications. For example, the coating compositions and methods may be used for drug delivery to a tissue surface, protection from damaging sources, antimicrobial and/or antiviral applications, pest repellent, an increase or reduction of friction between two surfaces, and/or ease of cleaning. Other examples of applications for the inventive coatings and methods are provided below. It should be understood that “coating composition” may refer to a coating on a surface or a composition for coating a surface.

In some embodiments, a coating composition comprises a plurality of components. Each component may impart a property to the coating composition. In some embodiments, the coating composition may include a polymer, an agent, and a solvent. The coating composition may also include components such as, but not limited to, gelling agents, fillers, excipients, etc., as described in more detail below. It should be understood that one of ordinary skill in the art may include other additives in the coating composition.

In some embodiments, the coating composition includes a polymer. A polymer may be the primary coating-forming material of the coating composition. A polymer may be degradable or non-degradable. A polymer may be obtained from natural sources or be created synthetically. In some embodiments, a polymer may comprise a methacrylate (i.e., butylmethacrylate, n-butylmethacrylate/iso-butylmethacrylate, ethyl methacrylate, Elvacite 2046, Elvacite 2028), acrylate, cellulose polymer, polyurethane, vinyl acetate, vinyl pyrrolidone polymer, silicone (i.e., fluids and waxes), copolymers thereof, and/or mixtures thereof. In some embodiments, a prepolymer and/or polymer precursor may be used to form a coating. For example, a coating may be formed by chemical reaction or physical interaction with one or more components delivered along with the prepolymer and/or polymer precursor, or by interaction with one or more components already present on the surface.

In some embodiments, the polymer may be a biodegradable polymer such as a polyester (i.e., polylactic acid, polyglycolic acid, polycaprolactone, etc.), polyanhydride, polycarbonate, and/or copolymers thereof.

The polymer may be a diblock copolymer, a triblock copolymer, etc., e.g., where one block is a hydrophobic polymer and another block is a hydrophilic polymer, or where both blocks are hydrophilic or both blocks are hydrophobic. In some embodiments, a coating composition includes a hydrophobic polymer, such as polymers that may include certain acrylics, amides and imides, carbonates, dienes, esters, ethers, fluorocarbons, olefins, styrenes, vinyl acetals, vinyl and vinylidene chlorides, vinyl esters, vinyl ethers and ketones, and vinylpyridine and vinylpyrrolidones polymers. In other embodiments, the coating composition includes a hydrophilic polymer, such as polymers including certain acrylics, amines, ethers, styrenes, vinyl acids, and vinyl alcohols. The polymer may be charged or uncharged. As noted herein, the particular components of the coating composition can be chosen so as to impart certain functionality to the structures.

In some embodiments, the polymer is essentially insoluble in water. The term “water-insoluble” is given its ordinary meaning in the art and refers to sparingly water-soluble, slightly, or very slightly water-soluble, and practically or totally water-insoluble compounds. In some cases, a water-insoluble polymer refers to a polymer whose solubility in water or an aqueous solution is less than 0.1 g/L at 25° C. In some cases, the solubility of the polymer is substantially independent from the acidity or basicity of the water or aqueous solution.

In some embodiments, a polymer may be modified to improve one or more properties. For example, a polymer may be crosslinked or lysed (i.e., hydrolyzed), or an existing crosslinking density may be increased or decreased. Such changes may be advantageous, for instance, for changing the degradation time of the polymer or the rate of release of an agent from the polymer. A polymer may be crosslinked, for example through covalent bonds, ionic bonds, hydrophobic bonds, and/or metal binding.

In some aspects, a polymer may be conjugated to an agent. In some embodiments, the agent may retain activity while conjugated. In other embodiments, the agent may be conjugated using a labile bond (i.e., a hydrolyzable bond) such that the agent may be released from the polymer in a controlled manner.

A coating composition may comprise a polymer in a concentration sufficient to form a coating with the desired barrier properties. Such concentrations can be determined readily by one of ordinary skill in the art. For example, a coating composition may comprise a polymer in an amount of 1 weight % to 40 weight %, 1 weight % to 20 weight %, 1 weight % to 15 weight %, 1 weight % to 10 weight %, 1 weight % to 6 weight %, 5 weight % to 20 weight %, 10 weight % to 30 weight %, or 20 weight % to 40 weight %. In preferred embodiments, an aerosol formulation may contain 1 weight % to 6 weight % of a polymer, a pump spray formulation may contain 1 weight % to 6 weight % of a polymer, a liquid formulation may contain 1 weight % to 20 weight % of a polymer, a wipe formulation may contain 1 weight % to 15 weight % of a polymer, a gel formulation may contain 1 weight % to 20 weight % of a polymer, and a lotion formulation may contain 1 weight % to 15 weight % of a polymer.

The coating may have any suitable thickness. For example, in some embodiments the coating may have a thickness between 1 mm and 100 microns, between 500 microns and 50 microns, between 100 microns and 10 microns, or between 50 microns and 1 micron. The coating may be thicker in some regions as compared to other regions. As a non-limiting example, when used as a skin barrier for a hand, the coating may be thicker on the palm than on the top of the hand (i.e., it may be advantageous to have a thicker coating on regions were more durability may be needed).

In some embodiments, a coating composition may be essentially colorless. An essentially colorless coating composition may be prepared by choosing suitable essentially colorless components. A coating composition may also be essentially transparent to visible light or may be translucent. The transparency or translucency of a coating composition may depend on properties such as the color of the components of the coating composition, the concentration of the components of the coating composition, or the thickness of the coating composition. These and other properties may be varied by one skilled in the art using routine experimentation until a suitable coating composition is found.

In some embodiments, the coating may form a barrier that is essentially impermeable to microorganisms for a period of time. For example, the barrier may be impermeable to microorganisms for at least 4 hours, at least 12 hours, at least 24 hours, at least 48 hours, or at least one week. It should be understood that even greater periods of time may be attainable.

In some aspects, a plurality of coatings may be applied to the skin. For example, a first coating may be applied, and one or more coatings may be applied on top of the first coating. A plurality of coatings may be used, for instance, to build up a barrier thickness sequentially. In other embodiments, at least two different coatings may be applied to the skin. For example, a first coating may contain a first agent intended for a first purpose, and a second coating, disposed on the first coating, may contain a second agent intended for a second coating. In some embodiments, at least one coating may contain an agent and at least one other coating may not contain an agent. For example, a first coating may be applied to the skin that does not contain a agent, and a second coating containing an agent may be applied on top of the first coating. Such a configuration may be used, for instance, to prevent the agent in the second coating from contacting the skin (i.e. to prevent absorption of an agent such as DEET through skin). In other embodiments, a first coating containing an agent may be applied to the skin and a second coating not containing an agent may be applied on top of the first coating. Such a configuration may be used, for instance, to protect the first coating, and/or prevent escape of the agent in the first coating.

In some embodiments, a coating may be formed from a plurality of particles. For instance, a plurality of particles may be suspended or dispersed within the coating composition and aggregate with one another on a surface to form a coating.

In some aspects, the coating composition may include a gelling agent. Examples of gelling agents include hydroxypropyl cellulose, hydroxypropyl methylcellulose, hydroxypropyl ethylcellulose, hydroxyethyl cellulose, hydroxymethyl cellulose, hydrocolloid gums, guar, acacia, tragacanth, xanthan, locust bean, carrageenan, clay thickeners, magnesium aluminum silicate, sodium magnesium silicate, alumina, bentonite, carbomer, polyox, and/or thickening polymers.

In some aspects, the coating composition is soluble in an organic solvent. Non-limiting examples of suitable organic solvents include alcohols (i.e., methanol, ethanol, isopropanol, butanol, pentanol, hexanol, isomers thereof, etc.), dimethylsulfoxide, N,N-dimethylformamide, N-methylpyrrolidone, hexamethyldisiloxane, gamma butyrolactone, glycol ethers, terpene solvents, ester solvents (i.e., ethyl acetate), acetone, mixtures thereof, and the like; however, any solvent that dissolves or suspends the polymer such that it can form a film on a surface may be used. Non-limiting specific examples of solvents include mixtures of isopropanol and N-methylpyrrolidone (i.e., for dissolving Elvacite 2046) and ethanol/N-methylpyrrolidone for dissolving Elvacite 2028.

In some embodiments, the coating composition is soluble in and/or comprises a volatile solvent. The term “volatile solvent” is given its meaning in the art and refers to a liquid having a measurable vapor pressure at atmospheric conditions (e.g., from about 0.01 mmHg to about 6 mmHg at 25° C.) and/or low boiling point (e.g., less than 25° C., or less than 200° C., or less than 150° C., or less than 100° C., or less than 90° C., or less than 80° C., or less than 70° C., or less than 60° C., or less than 50° C., or less than 40° C., or less than 30° C., or between room temperature and about 250° C., or between room temperature and about 200° C., or between room temperature and about 150° C., or between room temperature and about 100° C., or between room temperature and about 90° C., or between room temperature and about 80° C., or between room temperature and about 70° C., or between room temperature and about 60° C., or between room temperature and about 50° C., or between room temperature and about 40° C., or between room temperature and about 30° C.).

In some embodiments, the volatile solvent is an alcohol (e.g., methanol, ethanol, isopropanol, butanol, pentanol, hexanol, isomers thereof, combination thereof, etc.). In some embodiments, the volatile solvent is not an alcohol.

In some embodiments, the volatile solvent is a volatile silicone (e.g., hexamethyldisiloxane). The volatile silicon may be linear or cyclic, may comprise between 1 and 8 silicon atoms, and may further comprise C1-C10 and/or C1-C10 alkoxy groups. Non-limiting examples of volatile silicones include hexamethyldisiloxane, octamethylcyclotetrasiloxane, decamethylcyclopentasiloxane, dodecamethylcyclohexasiloxane, heptamethylhexyltrisiloxane, heptamethylethyltrisiloxane, heptamethyloctyltrisiloxane, octamethyltrisiloxane, decamethyltetrasiloxane, cyclomethicones (e.g., cyclotrisiloxane, cyclotetrasiloxane, cyclopentasiloxane, cyclohexasilxane, etc.), etc., or combinations thereof.

In some embodiments, the volatile solvent is a volatile ether (e.g., diethyl ether; dimethyl ether, dichlorodiethyl ether, propylene glycol monomethyl ether, propylene glycol monomethyl ether acetate, dipropylene glycol mono(n-butyl)ether, etc., or combinations thereof).

In some embodiments, the volatile solvent is a volatile ketone (e.g., acetone, N-methyl-2-pyrrolidone, methyl ethyl ketone, methyl isobutyl ketone, diisobutyl ketone, etc., or combinations thereof).

In some embodiments, the volatile solvent is a volatile ester (e.g., ethyl acetate, methyl acetate, propyl acetate, n-butyl acetate, isopentyl acetate, etc., or combinations thereof).

In some embodiments, the volatile solvent is a volatile aldehyde (e.g., benzaldehyde, acetaldehyde, etc., or combinations thereof).

The coating composition may comprise, in some embodiments, a solvent (e.g., organic and/or volatile) in a concentration between 40-95% by volume, between 70-90% by volume, between 60-80% by volume, between 60-95% by volume, between 60-90% by volume, between 80-95% by volume, between 50-70% by volume, between 40-60% by volume, or between 30-50% by volume. In a particular embodiment, the solvent (e.g., organic and/or volatile) is in a concentration between about 60% and about 95%. In another particular embodiment, the solvent (e.g., organic and/or volatile) is in a concentration between about 60% and about 90%. In yet another particular embodiment, the solvent (e.g., organic and/or volatile) is in a concentration between about 60% and about 80%.

In some embodiments, the concentration of solvent (e.g. organic and/or volatile) may be less than 60% by volume (e.g., between 1% and 60%, or between 10% and 60%, or between 20% and 60%, or between 30% and 60%, or between 40% and 60%, or between 50% and 60%, or between 1% and 50%, or between 10% and 50%, or between 20% and 50%, or between 30% and 50%, or between 40% and 50%, or between 10% and 40%, or between 20% and 40%). In other embodiments, the concentration of solvent (e.g. organic and/or volatile) may be less than 50% by volume.

In some embodiments, the coating composition may include an agent. An agent may be any entity that imparts a desirable property to a coating composition. In some embodiments, the agent may be selected from organic compounds, inorganic compounds, proteins, nucleic acids, and/or carbohydrates. In some embodiments, the agent may be a pharmaceutical agent. In certain aspects, the pharmaceutical agent may be used to treat the skin. For example, an agent may be an antimicrobial agent (i.e., antiviral, antibacterial, antifungal, etc.), an anti-acne agent, a corticosteroid (i.e., hydrocortisone, clobetasol propionate), nicotine, hormones, or anti-inflammatory compounds.

In some embodiments, an agent may be a skin protectant. Examples of general skin protectants include allantoin, dimethicone, zinc oxide, or zinc acetate. In other embodiments, the skin protectant may be a sunscreen, such as titanium dioxide, zinc oxide, avobenzone, octocrylene, octylmethoxycinnamate, or homosalate.

In still other embodiments, an agent may be a cosmetic. For example, an agent may be a hair treatment, moisturizer, skin brightener, skin radiance enhancer, anti-aging agent, and/or anti-wrinkling agent.

In yet further embodiments, an agent may be an insect repellent. Suitable insect repellents include, but are not limited to, DEET, picaridin, plant oils such as citronella, geraniol, lemon eucalyptus, neem, and the like, piperonyl butoxide, or pyrethrum.)

An agent may also be included that adds or withdraws heat from the skin. For example, spontaneous cooling agents or spontaneous heating agents may be included in a coating composition.

A skin coating may contain an agent at any suitable concentration. A simple test to determine a concentration at which to use an agent is to prepare a series of coating compositions containing an agent at various concentrations and apply them to a test surface such as the skin. An assay may be used to monitor the release and/or effectiveness or the agent over time. For example, in embodiments where the agent is an antimicrobial, the type and/or quantity of organisms on the test surface may be monitored using techniques known in the art such as microscopy, quantitative PCR, culturing, etc. Based on the results of the assay, the agent concentration may be adjusted in order to achieve the desired effectiveness. In some embodiments, the concentration of an agent may be between 1% and 30%, between 20% and 30%, between 15% and 25%, between 10% and 20%, between 5% and 15%, between 1% and 10%, between 0.1% and 5%, between 0.01% and 1%, between 0.1% and 2%, or between 0.5% and 2%. It should be understood that concentrations outside these ranges may also be used. In one example, a skin protectant may contain 0.5%-2% allantoin, 1%-30% dimethicone, 1%-25% zinc oxide, and 0.1%-2% zinc acetate.

As discussed in more detail below, a coating composition may contain a particle formulation configured for sustained release of an agent. A particle formulation may be prepared and/or modified according to methods known in the art to control the release rate of an agent.

A coating composition may contain an emulsion barrier delivery system. For example, a coating material selected from hydrophobic materials such as silicone compounds (i.e., high molecular weight silicone fluids and waxes), natural and/or synthetic waxes (beeswax, carnauba, candelilla, paraffin), petrolatum oil, mineral oil, fatty acids, alcohols, natural oils, synthetic oils, and/or blends thereof may be used to create emulsions containing agents. The hydrophobic materials may be used in place or in addition to the polymeric materials described elsewhere herein. An emulsion barrier may include an oil phase comprising, for example, one or more of the hydrophobic materials listed above, a water phase, and an agent that provides essentially continuous effects while the barrier is disposed on the skin. Without wishing to be bound by any theory, it is believed that after applying an emulsion to the skin, the emulsion breaks and the aqueous component evaporates resulting in the formation of a coating on the skin. An emulsion may be prepared using methods such as homogenation, sonication, and/or other methods known to those skilled in the art. In some embodiments, an emulsifier may be added to the coating composition to aid the formation of an emulsion. Examples of emulsifiers include surfactants, lecithin, polyvinylalcohol, detergents, cetearyl alcohol, polysorbate 20, ceteareth 20, and/or other emulsifiers known to those skilled in the art. A barrier formed from an emulsion may be removed by any suitable method, for example using an aqueous detergent solution (i.e., soap and water).

Coating compositions of the present invention may be used in any suitable application. For example, the coating composition may have antimicrobial and/or antiviral properties, which may be used in settings such as households, hospitals, clinician offices, food services, schools and daycares, nursing homes, gyms and health clubs, janitorial services, and/or pools or spas. A coating composition may also be used as a skin protectant spray, a first aid for temporary protection of minor cuts, scrapes, burns, etc., and/or a hand sanitizer with residual germ-killing strength. In another example, the coating composition may be used for sun protection. Further still, the coating composition can be used for controlled release of an agent (i.e., pharmaceutical, prophylactic, and/or cosmetic). In some aspects, the coating composition may be used as a friction barrier, for example to reduce heat generation between two rubbing surfaces. The friction barrier may also be used for anti-chafing purposes (i.e., for runners and/or cyclists, in gyms and health clubs, and/or for general foot care). In some aspects, the coating compositions may be used as a soil barrier. For instance, the coating may facilitate removal of oil and/or dirt from a surface (i.e., from the skin during and/or after gardening, camping, mechanical device repair, painting, and/or construction). The coatings may also be used as a chemical barrier, for instance in households, janitorial services, and/or laboratories. In some aspects, the coatings may be used as an insect barrier. For example, the coating may contain an agent that repels insects. When formed on skin, the coating may physically prevent insect bites. In another embodiment, the coating may prevent plant oils such as poison ivy, poison oak, and/or poison sumac from contacting the skin. The coating may also operate as a protective shield for cracked skin and/or blisters. In yet another embodiment, the coating may reduce the slipperiness of a surface (i.e., improve the grip between a hand and/or foot and a surface). A coating may also be used to shield a surface from aqueous solutions (i.e., water sports and/or dishwashing).

Further desirable properties of the coating compositions include a fast-drying formulation, long life (i.e., durable), minimal or absent tackiness, and ability to be removed on demand (i.e., using a suitable solvent, as describe in more detail below). For example, in some embodiments, coating composition may be dry in less than 5 seconds, less than 10 seconds, less than 30 seconds, or less than 1 minute. The coating may be durable enough to remain substantially intact during, for example, physical activities and/or exposure to sweat. The speed at which a coating dries may be controlled, for example, by the type of solvent used to apply the coating. For example, the use of lower molecular weight solvents (e.g., alcohols) that are more volatile than other solvents (e.g., alcohols) may result in faster drying, whereas the use of higher molecular weight solvents (e.g., alcohols) that are generally less volatile may result in slower drying. The rate of drying may be important, for example, for the antiseptic effect of the solvent.

Additionally, a coating composition may have desirable characteristics such as reducing the slipperiness of skin, being waterproof, being soap and water resistant, having stability over prolonged periods of time (i.e., greater than 1 week, greater than 1 month, or greater than 3 months).

In a preferred embodiment, a coating composition has an essentially immediate antiseptic effect, a residual microbiostatic and/or microbiocidal effect (i.e., sustained antisepsis), and a resilient barrier which shields skin from exposure and, in some embodiments, locks active ingredients in place.

In some embodiments, the essentially immediate antiseptic effect is provided by a quaternary amine (i.e., benzalkonium chloride). In some embodiments, the essentially immediate antiseptic effect is provided by an alcohol and/or a quaternary amine (i.e., benzalkonium chloride). In some embodiments, the efficacy of the essentially immediate antiseptic effect may be greater than 99.9% of microorganisms killed within 5 seconds, within 15 seconds, within 30 seconds, or within 1 minute. Other measurements of antiseptic efficacy are well known in the art. For example, the United States Food and Drug Administration (“FDA”) has published proposed test methods and performance requirements for healthcare disinfectants, including skin-preparations. The FDA's proposed methods and performance requirements are described in the “Tentative Final Monograph for Health-Care Antiseptic Drug Products” (the “TFM”). Such performance requirements include the TFM “Time-Kill” test, the TFM “Minimum Inhibition Concentration” test, and the TFM “Efficacy” test. Other performance tests may also be used.

The residual microbiostatie and/or microbiocidal effect may be provided by any suitable agent, examples of which are described below. In some embodiments, the residual effect may last for at least 4 hours, at least 12 hours, at least 24, or longer. In some aspects, the agent may be active immediately upon formation of a coating. In some embodiments, an agent may provide a prophylactic activity. For example, an agent may have microbiostatic properties that may prevent new organisms from growing on a surface but may not be able to sanitize a surface. In other embodiments, the agent may be capable of sanitizing a surface.

Coating compositions of the present invention may be applied by any suitable method. For example, a coating composition may be applied using an aerosol, pump spray, foam, wipe, dip, liquid, gel, lotion, and/or cream. Methods for preparing formulations suitable for application using such techniques are well known to those skilled in the art. As described above, the coating compositions may be applied using a solvent that dries quickly (i.e., is volatile). In some embodiments, an activator may be applied which speeds the drying and/or curing of the coating composition.

The coating composition may be maintained on the skin for an extended period of time. However, in some embodiments, it may be desirable to remove the coating, for example, when no longer needed, or when effectiveness falls below a threshold value. In some embodiments, it may be desirable to remove a coating, or what remains of a coating, prior to reapplication of a coating. In some embodiments, a coating may be removed using a suitable solvent. For example, an alcohol or other solvent as described above may be used to dissolve the coating, thus allowing the coating composition to be wiped away or washed away. In other embodiments, the coating may be removed by degradation, such as by a degrading entity. For example, a coating may be acid labile, base labile, enzymatically labile, and/or light labile. Exposure of a coating to a degrading entity may cause, in some embodiments, breakdown of the coating composition into fragments that may be washed away, may be soluble in water, and/or may be rubbed off.

Those of ordinary skill in the art can chose appropriate materials to control the rates of degradation of the material after it has been delivered to the subject. For instance, the polymer in a particulate or other form may substantially or completely degrade within the subject after at least one day, at least three days, at least one week, at least two weeks, at least one month, at least six months, or at least one year. The rate of degradation will depend on the condition to be treated among other factors.

In some aspects, a coating composition may be used to deliver an agent through the skin. In some embodiments, an agent released from the coating may be absorbed through the skin. In other embodiments, a suitable carrier may be used to facilitate transdermal delivery. Suitable pharmaceutical compositions and methods are described in more detail below. In some embodiments, an agent may be released in a sustained fashion as described in more detail below.

The polymers and particles described herein may be used in “pharmaceutical compositions” or “pharmaceutically acceptable” compositions, which comprise a therapeutically effective amount of an active agent associated with one or more of the coating compositions described herein, formulated together with one or more pharmaceutically acceptable carriers, additives, and/or diluents. The pharmaceutical compositions described herein may be useful for diagnosing, preventing, treating or managing a disease or bodily condition including cardiac and certain vascular conditions.

The pharmaceutical compositions may be specially formulated for administration in gel or liquid form, including those adapted for the following: a sterile solution or suspension, a sustained-release formulation, or as a cream or foam. In some embodiments, a composition includes one or more polymers dissolved and/or suspended in a solvent that form a coating on skin.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid, gel or solid filler, diluent, excipient, or solvent encapsulating material, involved in carrying or transporting the subject compound, e.g., from a device or from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The amount of active agent which can be combined with a coating composition to produce a single dosage form will vary depending upon the host being treated, and the particular mode of administration. The amount of active agent that can be combined with a coating composition to produce a single dosage form will generally be that amount of the compound which produces a therapeutic effect. Generally, this amount will range from about 1% to about 99% of active ingredient, from about 5% to about 70%, or from about 10% to about 30%.

Coating compositions described herein suitable for forming a coating on skin may be administered in the form of a solution, dispersion, or a suspension in an aqueous or non-aqueous liquid, as an emulsion or microemulsion (e.g., an oil-in-water or water-in-oil liquid emulsion), or as an elixir or syrup, or as pastilles (using an inert base, such as gelatin and glycerin, or sucrose and acacia), each containing a predetermined amount of a coating material described herein, and optionally including an active ingredient.

Examples of suitable aqueous and nonaqueous carriers, which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of other coating materials, such as lecithin, by the maintenance of the required particle size in the case of dispersions, and by the use of surfactants.

In addition to the coating materials, a liquid dosage form may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the coating materials, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

These coating compositions described herein may also contain adjuvants such as preservatives, wetting agents, emulsifying agents, lubricating agents and dispersing agents. Prevention of the action of microorganisms on and/or in the coating compositions may be facilitated by the inclusion of various antibacterial and antifungal agents, for example, paraben, chlorobutanol, phenol sorbic acid, and the like. It may also be desirable to include isotonic agents, such as sugars, sodium chloride, and the like into the coating compositions. In addition, prolonged absorption of the injectable pharmaceutical form may be brought about by the inclusion of agents which delay absorption such as aluminum monostearate and gelatin.

Antimicrobial agents include wide spectrum antimicrobials, narrow spectrum antimicrobials, zinc oxide (i.e., colloidal), silver compounds (i.e., colloidal), triclosan, triclocarban, parachlorometaxylenol (PCMX), polyhexamethylene biguanide (PHMB), quaternary ammonium compounds (i.e., benzalkonium chloride, benzethonium chloride, etc.), cetrimonium chloride, domiphen bromide, chlorohexidine gluconate, phenylmercuric salts (i.e., borate, acetate, etc.), phenol-type antimicrobial agents, povidone-iodine, iodophors, parabens, hyantoins, isothiazolinones, iodopropynyl butylcarbamate, 1-(3-chloroallyl)-3,5,7-triaza-1-azoniaadamantane chloride, benzoates, sorbates, propionates, and the like.

Antifungal agents include miconazole, tolnaftate, clioquinol, haloprogin, miconazole nitrate, povidone-iodine, tolnaftate, undecylenic acid and related salts (i.e., calcium, copper, zinc, etc.), clotrimazole, and the like.

Delivery systems suitable for use with polymers, particles and compositions described herein include time-release, delayed release, sustained release, or controlled release delivery systems. Such systems may avoid repeated administrations of the particles and/or active agents in many cases, increasing convenience to the subject and the physician. Many types of release delivery systems are available and known to those of ordinary skill in the art. Specific examples include, but are not limited to, erosional systems in which the composition is contained in a form within a matrix, or diffusional systems in which an active component controls the release rate. The compositions may be as, for example, particles (e.g., microparticics, microspheres), hydrogels, polymeric reservoirs, or combinations thereof. In some embodiments, the system may allow sustained or controlled release of an active agent to occur, for example, through control of the diffusion or erosion/degradation rate of the formulation or particle. The polymers, particles and compositions described herein can also be combined (e.g., contained) with delivery devices such as syringes, catheters, tubes, and implantable devices.

Use of a long-term release coating may be particularly suitable in some embodiments. “Long-term release,” as used herein, means that the coating is constructed and arranged to deliver therapeutic levels of the composition for at least about 30 or about 45 days, for at least about 60 or about 90 days, or even longer in some embodiments. Long-term release coatings are well known to those of ordinary skill in the art. In some embodiments, a long-term release coating can be formed by coating at least a portion of the skin of a subject, after which the coating remains on the subject for an extended period.

When the coating compositions described herein are administered as pharmaceuticals, to humans and animals, they can be given per se or as a pharmaceutical composition containing, for example, about 0.1% to about 99.5%, about 0.5% to about 90%, or the like, of coating composition in combination with a pharmaceutically acceptable carrier.

The pharmaceutical compositions described herein may be given in dosages, e.g., at the maximum amount while avoiding or minimizing any potentially detrimental side effects. The pharmaceutical compositions can be administered in effective amounts, alone or in a combinations with other compounds. For example, when treating cancer, a pharmaceutical composition may include the coating compositions described herein and a cocktail of other compounds that can be used to treat cancer.

The phrase “therapeutically effective amount” as used herein means that amount of a material or composition comprising an inventive structure which is effective for producing some desired therapeutic effect in a subject at a reasonable benefit/risk ratio applicable to any medical treatment. Accordingly, a therapeutically effective amount may, for example, prevent, minimize, or reverse disease progression associated with a disease or bodily condition. Disease progression can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

The effective amount of a coating composition described herein may be from about 10 ng/kg of body weight to about 1000 mg/kg of body weight, and the frequency of administration may range from once a day to a once a month basis, to an as-needed basis. However, other dosage amounts and frequencies also may be used as the invention is not limited in this respect. A subject may be administered one or more coating compositions described herein in an amount effective to treat one or more diseases or bodily conditions described herein.

The effective amounts will depend on factors such as the severity of the condition being treated; individual patient parameters including age, physical condition, size and weight; concurrent treatments; the frequency of treatment; or the mode of administration. These factors are well known to those of ordinary skill in the art and can be addressed with no more than routine experimentation. In some embodiments, a maximum dose be used, that is, the highest safe dose according to sound medical judgment.

The selected dosage level can also depend upon a variety of factors including the activity of the particular inventive structure employed, the route of administration, the time of administration, the rate of excretion or metabolism of the particular particles or active agents being employed, the duration of the treatment, other drugs, compounds and/or materials used in combination with the particular coating composition employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the structures described herein employed in the pharmaceutical composition at levels lower than that required to achieve the desired therapeutic effect and then gradually increasing the dosage until the desired effect is achieved.

In some embodiments, a coating or pharmaceutical composition described herein is provided to a subject chronically. Chronic treatments include any form of repeated administration for an extended period of time, such as repeated administrations for one or more months, between a month and a year, one or more years, or longer. In many embodiments, a chronic treatment involves administering a coating or pharmaceutical composition repeatedly over the life of the subject. For example, chronic treatments may involve regular administrations, for example one or more times a week, or one or more times a month.

While it is possible for a coating composition described herein to be administered alone, it may be administered as a pharmaceutical composition as described above. The present invention also provides any of the above-mentioned compositions useful for diagnosing, preventing, treating, or managing a disease or bodily condition packaged in kits, optionally including instructions for use of the composition. That is, the kit can include a description of use of the composition for participation in any disease or bodily condition. The kits can further include a description of use of the compositions as discussed herein. Instructions also may be provided for administering the composition by any suitable technique.

The kits described herein may also contain one or more containers, which can contain components such as the polymer, active agent, and/or volatile solvent (e.g., alcohol) as described herein. The kits also may contain instructions for mixing, diluting, and/or administrating the components. The kits also can include other containers with one or more solvents, surfactants, preservatives, and/or diluents (e.g., normal saline (0.9% NaCl), or 5% dextrose) as well as containers for mixing, diluting or administering the coating compositions to the patient in need of such treatment.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are used, the liquid form may be concentrated or ready to use. The solvent will depend on the particular coating composition and the mode of use or administration. Suitable solvents for compositions are well known and are available in the literature.

The kit, in one set of embodiments, may comprise one or more containers such as vials, tubes, syringes, and the like, each of the containers comprising one or more of the elements to be used in the method. For example, one of the containers may contain a solution or suspension of particles. Additionally, the kit may include containers for other components, for example, buffers or diluents to be mixed with the particles prior to delivery.

As used herein, a “subject” or a “patient” refers to any mammal (e.g., a human), for example, a mammal that may be susceptible to a disease or bodily condition. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition.

These above descriptions of applications for the inventive compositions and methods devices are not intended to be exhaustive, and merely illustrate some of the possible embodiments and uses of this invention.

The function and advantage of these and other embodiments of the present invention may be more fully understood from the examples below. The following examples, while illustrative of certain embodiments of the invention, do not exemplify the full scope of the invention.

### EXAMPLES

### Example 1

This example provides antimicrobial coating composition formulations of the present invention.

Composition 1 includes 50:50 n-butyl/isobutyl methacrylate copolymer (5.0+/−1.0% by weight), isopropanol (70.0+/−20.0% by weight), hexamethyldisiloxane (40.0+/−10.0% by weight), and benzalkonium chloride (0.025+/−0.015% by weight).

Composition 2 includes 50:50 n-butyl/isobutyl methacrylate copolymer (5.0+/−1.0% by weight), isopropanol (70.0+/−20.0% by weight), hexamethyldisiloxane (40.0+/−10.0% by weight), and triclosan (0.2+/−0.1% by weight).

Composition 3 includes ethyl methacrylate copolymer (5.0+/−1.0% by weight), ethanol (70.0+/−20.0% by weight), hexamethyldisiloxane (40.0+/−10.0% by weight), and benzalkonium chloride (0.025+/−0.015% by weight).

Composition 4 includes ethyl methacrylate copolymer (5.0+/−1.0% by weight), ethanol (70.0+/−20.0% by weight), hexamethyldisiloxane (40.0+/−10.0% by weight), and triclosan (0.2+/−0.1% by weight).

### Example 2

This example provides a sunscreen coating composition formulation of the present invention.

An SPF 25 sunscreen coating formulated as a spray was prepared according to the following table.

| | Concentration | Concentration |
|---|---|---|
| | without | with propellant |
| Ingredient | propellant (w/w %) | (w/w %) |
| isopropanol | 56.4 | 42.3 |
| octocrylene | 13.3 | 10.0 |
| oxybenzone | 6.7 | 5.0 |
| avobenzone | 3.3 | 2.5 |
| Elvacite 2046 (50/50 n-butyl/isobutyl | 4.0 | 3.0 |
| methacrylate) | | |
| hexamethyldisiloxane | 13.3 | 10.0 |
| Shinetsu KP-545 | 0.8 | 0.6 |
| DC200, 350cs (dimethicone) | 1.3 | 1.0 |
| Citroflex 2 (triethyl citrate) | 0.8 | 0.6 |
| A-46 (propellant) | — | 25.0 |
| Total | 100 | 100 |

### Example 3

This example provides an antifungal coating composition formulation of the present invention.

An antifungal coating formulated as a spray was prepared according to the following table.

| | Concentration | Concentration |
|---|---|---|
| | without propellant | with propellant |
| Ingredient | (w/w %) | (w/w %) |
| isopropanol | 55.6 | 41.7 |
| Tolnaftate, USP | 1.7 | 1.28 |
| Elvacite 2046 (50/50 n-butyl/isobutyl | 4.0 | 3.0 |
| methacrylate) | | |
| hexamethyldisiloxane | 35 | 26.25 |
| Shinetsu KP-545 | 1 | 0.75 |
| DC200, 350cs (dimethicone) | 1.7 | 1.28 |
| Citroflex 2 (triethyl citrate) | 1 | 0.75 |
| A-46 (propellant) | — | 25.0 |
| Total | 100 | 100 |

### Example 4

This example provides an insect repellent coating composition formulation of the present invention.

An insect repellent coating formulated as a spray was prepared according to the following table.

| | Concentration | Concentration |
|---|---|---|
| | without propellant | with propellant |
| Ingredient | (w/w %) | (w/w %) |
| isopropanol | 47.95 | 35.96 |
| Picaridin | 9.35 | 7.01 |
| Elvacite 2046 (50/50 n-butyl/isobutyl | 4.0 | 3.0 |
| methacrylate) | | |
| hexamethyldisiloxane | 35 | 26.25 |
| Shinetsu KP-545 | 1 | 0.75 |
| DC200, 350cs (dimethicone) | 1.7 | 1.28 |
| Citroflex 2 (triethyl citrate) | 1 | 0.75 |
| A-46 (propellant) | — | 25.0 |
| Total | 100 | 100 |

### Example 5

This example provides an antifugal coating composition formulation of the present invention comprising an alcohol, a volatile silicone, and a ketone.

An antifugal coating composition was prepared according to the following table.

| | | BULK |
|---|---|---|
| Ingredient | Exemplary Trade Name | % w/w |
| Ethanol, specially denatured | SDA 40B 200 | 50.0 |
| Methacrylate Copolymer | Elvacite 2028 | 4.0 |
| Tolnaftate, USP | Tolnaftate, USP | 1.3 |
| Hexamethyldisiloxane | Hexamethyldisiloxane | 40.0 |
| Acrylates/dimethicone copolymer | Shinetsu KP-546 | 0.7 |
| Triethyl citrate | Citroflex 2 | 1.0 |
| N-Methyl 2-Pyrrolidone | M-Pyrol | 3.0 |
| Propellant | A-46 | — |
| Total | | 100.0 |

### Example 6

This example provides an antibacterial barrier film gel coating composition formulation of the present invention comprising a volatile silicone and a ketone.

An antibacterial barrier film gel coating composition was prepared according to the following table.

| Ingredient | Exemplary Trade Name | % w/w |
|---|---|---|
| Hexamethyldisiloxane | Hexamethyldisiloxane | 89.75 |
| Methacrylate Copolymer | Elvacite 2028 | 5.00 |
| Hydroxypropylcellulose | Klucel M | 2.00 |
| Acrylates/dimethicone copolymer | Shinetsu KP-546 | 1.00 |
| Dimethicone | DC200, 1000cps | 1.00 |
| Triethyl citrate | Citroflex 2 | 1.00 |
| Tea Tree Oil | Tea Tree Oil | 0.10 |
| Silver compound | Acticare | 0.05 |
| Fragrance | Fragrance | 0.10 |
| Total | | 100 |

While several embodiments of the invention have been described and illustrated herein, those of ordinary skill in the art will readily envision a variety of other means and structures for performing the functions and/or obtaining the results or advantages described herein, and each of such variations, modifications and improvements is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention.

In the claims (as well as in the specification above), all transitional phrases or phrases of inclusion, such as “comprising,” “including,” “carrying,” “having,” “containing,” “composed of,” “made of,” “formed of,” “involving” and the like shall be interpreted to be open-ended, i.e., to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.”

All references cited herein, including patents and published applications, are incorporated herein by reference. In cases where the present specification and a document incorporated by reference and/or referred to herein include conflicting disclosure, and/or inconsistent use of terminology, and/or the incorporated/referenced documents use or define terms differently than they are used or defined in the present specification, the present specification shall control.

What is claimed is:

1. A composition, comprising:
an essentially water-insoluble polymer;
an agent; and
a volatile solvent, wherein the polymer and agent form an essentially colorless and transparent coating on a surface upon evaporation of the volatile solvent, wherein the coating is essentially impermeable to microorganisms, permeable to moisture vapor, and configured for controlled release of the agent, wherein the volatile solvent is initially present at a concentration between 60% and 95% by volume.

2. The composition of claim 1, wherein the polymer comprises butyl methacrylate.

3. The composition of claim 1, wherein the polymer comprises ethyl methacrylate.

4. The composition of claim 1, wherein the polymer comprises a copolymer of n-butyl methacrylate and iso-butyl methacrylate.

5. The composition of claim 1, wherein the polymer is flexible.

6. The composition of claim 1, wherein the volatile solvent comprises ethanol

7. The composition of claim 1, wherein the volatile solvent comprises isopropanol.

8. The composition of claim 1, wherein the volatile solvent comprises a volatile silicone, a volatile ether, a volatile ketone, a volatile ester, a volatile aldehyde, or a violative alcohol, or a combination thereof.

9. The composition of claim 1, wherein the volatile solvent is not an alcohol.

10. The composition of claim 1, wherein the agent comprises an antimicrobial.

11. The composition of claim 8, wherein the antimicrobial comprises benzalkonium chloride.

12. The composition of claim 11, wherein the antimicrobial comprises triclosan.

13. The composition of claim 1, wherein the polymer, agent, and alcohol solvent are formulated as a liquid, foam, spray, wipe, gel, lotion, or cream.

14. The composition of claim 1, wherein the coating is essentially impermeable to microorganisms for at least 24 hours.

15. The composition of claim 1, wherein the coating is essentially impermeable to microorganisms for at least 48 hours.

16. The composition of claim 1, further comprising dimethicone.

17. The composition of claim 1, further comprising hexamethyldisiloxane.

18. A composition, comprising:
an essentially water-insoluble polymer;
an agent;
a quaternary amine antiseptic; and
a volatile solvent, wherein the polymer and agent form an essentially colorless and transparent coating on a surface upon evaporation of the volatile solvent, wherein the coating is essentially impermeable to microorganisms, permeable to moisture vapor, and configured for controlled release of the agent, wherein the volatile solvent is initially present at a concentration less than 60% by volume.

19. The composition of claim 18, wherein the volatile solvent is not an alcohol.

20. A method, comprising:
contacting a surface with a composition comprising a polymer, an active agent, and a volatile solvent, wherein the polymer and active agent form an essentially colorless and transparent coating on the surface upon evaporation of the volatile solvent, the composition sanitizes the surface essentially immediately, and the coating is capable of sustained antisepsis.

21. The method of claim 20, wherein the volatile solvent is not an alcohol.
